(19) United States
(12) Patent Application Publication
(10) Pub. No.: US 2005/0203429 A1
(43) Pub. Date: Sep. 15, 2005

(54) Device and method for measuring cardiac function

(75) Inventor: William V. Judy, Bradenton, FL (US)

Correspondence Address: BROWDY AND NEIMARK, P.L.L.C.;624 NINTH STREET, NW, SUITE 300, WASHINGTON, DC, 20001-5303, US

(73) Assignee: IMPEDANCE VASCULAR IMAGING PRODUCTS, LLC, Bradenton, FL (US)

(21) Appl. No.: 10/800,004

(22) Filed: Mar. 15, 2004

(51) Int. Cl. A61B 5/04

(52) U.S. Cl. 600/508; 600/547

(57) ABSTRACT

A device and method for determining at least the left ventricular end diastolic volume LVEDV of a beating heart of in particular a human being, is disclosed, the device comprising a current source supply electrodes, for supplying alternating current to the body, measuring electrodes, for receiving an impedance signal, measuring means, for measuring said impedance signal, a processing unit, for processing and outputting the value of said impedance signal and the first time derivative thereof, and means for determining the duration DFT of a heartbeat, and wherein the processing unit is designed for determining and outputting the value of LVEDV in dependence of both said duration DFT and said impedance and the change of the first time derivative of said impedance during the pre-ejection period.

### FIELD OF THE INVENTION

The present invention generally relates to a device and method for measuring quantities related to cardiac function. More particularly, the present invention is directed to the use of a bioimpedance device and method to determine non-invasively and on a beat-to-beat basis the left ventricular end diastolic volume and other quantities of the animal or human heart.

### BACKGROUND OF THE INVENTION

Cardiac monitoring is the measuring of various quantities related to the functioning of the human or animal heart. Some of the measured quantities are heart rate (HR), stroke volume (SV), cardiac output (CO=HR×SV), etc. A number of other quantities, which are used in this application, are to be understood as follows:
left ventricular end diastolic volume (LVEDV): the maximum volume of the left ventricle, just before the contraction of the heart.
left ventricular end systolic volume (LVESV): the minimum volume of the left ventricle, at the end of the contraction of the heart, LVESV=LVEDV−SV.
Ejection fraction (EF), which is equal to stroke volume divided by (left ventricular) end diastolic volume (SV/LVEDV). Ejection fraction is a measure of the pumping efficiency of the heart, and notably of the left ventricle.

In the art, various methods are employed to determine one or more of the above-mentioned quantities. One such known method is thermodilution. Herein one injects a fluid, often water, of a certain temperature into the right ventricle. Then one measures the temperature of the diluted fluid in the pulmonary artery. From this the right ventricular stroke volume may be determined. On average this will be equal to the left ventricular stroke volume, but on a beat-to-beat basis there may be differences. Furthermore, the diluted fluid is measured only during the first five or six seconds after the beat, after which the rest of the curve is extrapolated, which may lead to errors. Moreover, it is an invasive and non-continuous method, with relatively high risk and discomfort for the patient. Furthermore, this method requires expert medical attention. A comparable method is the dye dilution method, in which a dye is injected, and its concentration in the blood is measured.

Another method known in the art is X-ray ventriculography, in which a contrast fluid is injected into the left ventricle. With the aid of 3-dimensional X-photographs a picture of the heart can be obtained. This method can measure left ventricular quantities, but requires a catheter to be placed in the left ventricle via the aorta. Hence, this is also an invasive method, which is very risky and bothersome for the patient, is laborious and costly and is furthermore rather inaccurate because relatively crude geometrical models must be used to calculate the quantities. A comparable method is nuclear cardiography, wherein an radioactive isotope is injected and detected.

Some known non-invasive methods to measure a heart related quantity, like LVEDV, SV, cardiac output, diastolic filling time or EF; are echocardiography, which uses the echo of ultrasound waves to determine blood flows, the Fick method (for cardiac output) and the CO2 rebreathing method. Neither of the above methods is very accurate and versatile. For determining the diastolic filling time, there exist many invasive methods, like left ventricular pressure tracings or left ventricular volume change tracings, and non-invasive methods, like phonocardiography, electrocardiography, and carotid pulse signals. All these methods have the inherent error of inclusing the isovolumetric period.

One other known method is the method of (transthoracic) bio electrical impedance measurement, or impedance plethysmography, which was described in U.S. Pat. No. 3,340,867 to Kubicek et al. This method is non-invasive, safe for the patient and continuous. In the method, an alternating current field is established in the thorax of a patient with the help of two supply electrodes, one around the neck and one around the torso, below the thorax. Between two measuring electrodes, both between the two supply electrodes at slightly smaller mutual distance, a (voltage) signal is measured, which is indicative of the impedance of all the tissue in between, i.e. the lungs, the heart and blood vessels. More precisely, pulsatile impedance changes that correlated with heartbeat, were found to be due to blood displacement from the left ventricle to the aorta. It was found that the stroke volume (SV) could be determined from this signal, the formula being:
SV=ρ(L/Z₀)²×(VET)(dZ/dT)ₘₐₓ
wherein
ρ=resistivity of the blood,
L=distance between supply electrodes,
Z₀=basic impedance between the measuring electrodes,
VET=ventricular ejection time, i.e. the time between opening and closing of the aortic valve, and
(dZ/dT)ₘₐₓ=maximum value of the first time derivative of the impedance value Z during the VET.

This method had the above-mentioned advantages, but it showed some inaccuracy when compared to the standard thermodilution method. In part this was caused by the rather crude model that was used, e.g. for the thorax. Also, thermodilution is a kind of averaging method, while the bioimpedance method is on a beat-to-beat-basis. This makes comparison even more difficult. Furthermore, it could not determine ejection fraction nor LVEDV.

In an article called “Relationship between bioimpedance, thermodilution and ventriculographic measurements in experimental congestive heart failure” by Spinale et al., in “Cardiovascular Research”, 1990, 24, page 423-429, an improved formula for SV was mentioned, that was based on a model by Bernstein, and furthermore, an indirect relationship was found between ejection fraction (EF) and (dZ/dT)ₘₐₓ, viz.
EF=13.2×(dZ/dT)ₘₐₓ+19.3 [%] (for pigs with induced tachycardia).

With the help of this formula it is possible to determine LVEDV, which is equal to SV/EF.

Although it might be expected that a similar relationship would hold for all mammalian hearts including the human heart, still such relationship would depend on a determination of SV, and of (dZ/dT)ₘₐₓ as well. The margin of error for both variables would have to be combined. Furthermore, the formula for determining EF was found to be inaccurate (standard error=11%) and not to give very useful results.

Therefore there exists a need for a device and method to determine LVEDV, and preferably EF as well, safely, non-invasively, continuously and accurately.

Moreover, there is a need to improve the accuracy of the existing bioimpedance method to a clinically acceptable level.

### SUMMARY OF THE INVENTION

One aspect of the present invention is a device for determining at least a left ventricular end diastolic volume LVEDV for a heartbeat of a beating heart of a mammal, in particular a human being, having a body having a thorax. The device comprises a current source for producing an alternating current with a frequency, one or more upper supply electrodes and one or more lower supply electrodes, which can be applied to the body of the mammal and which can be connected to the current source, for supplying the alternating current to the body,

one or more upper measuring electrodes and one or more lower measuring electrodes, which can be applied to the body of the mammal above the heart, below the heart, respectively, for receiving an impedance signal that depends on the impedance of a part of the thorax at least comprising the heart,

measuring means, connected to the measuring electrodes, for measuring the impedance signal which is received by the measuring electrodes, a processing unit, connected to the measuring means, for processing and outputting at least the value of the impedance signal and the first time derivative of the impedance signal,

wherein the device further comprises means for determining the duration DFT of a diastolic filing time of the heart during said heartbeat and for determining the value of the impedance signal at the end of a pre-ejection period of said heart during said heartbeat, which diastolic filing time and pre-ejection period may be determined in a manner known per se, and wherein the processing unit is designed for determining and outputting the value of the left ventricular end diastolic volume LVEDV in dependence of both the duration DFT and said value of the impedance signal at the end of said pre-ejection period and the difference of the value of the first time derivative of the impedance signal between the beginning and said end of said pre-ejection period.

Preferably, the means for determining said duration DFT comprise an electronic circuit, which is able to determine said duration DFT as the time between the moment at which said first time derivative assumes a third local minimum value following a maximum value during said heartbeat, and the moment at which said first time derivative assumes a minimum value immediately before the next maximum value. The diastolic filling time DFT runs from the opening of the mitral valve to the next closure of the mitral valve. The opening of the mitral valve is marked by the beginning of the O-wave of the dZ/dt-signal. This O-wave begins with the third local minimum after the absolute maximum value of the dZ/dt-signal. The closure of the mitral valve is marked by the deep negative peak just before the next maximum value of the next heartbeat. These maximum and minimum values will be further elucidated in the description of the preferred embodiment. Relatively simple electronics and/or computer programming may provide excellent accuracy to determine DFT.

In another preferred embodiment, the means for determining said duration DFT are sound recording means which are able to determine said duration DFT as the time between the beginning of the second component of the heartsound following a maximum value of said first time derivative during said heartbeat and the beginning of the next heartsound. These heartsounds will be elucidated in the description of the preferred embodiment. By thus using acoustic signals, there is no risk of electrical interference with other signals. However, heartsounds are less reliable as a means for the unambiguous determination of DFT. Hence other methods may be used, such as for example a measurement of DFT based on the diastolic component of the carotid pulse, EKG or other ejection-related phenomena. However, with these other methods it is more difficult to measure DFT, that is, the diastolic time interval minus the isovolumetric relaxation period, to be described later on. Hence these other methods might give an error of about 10%.

Advantageously, the processing unit outputs said left ventricular end diastolic volume LVEDV as

$$LVEDV = C \cdot {( \frac{L}{{M}_{0}} )}^{2} \cdot DFT \cdot Δ \frac{ⅆ {Z}_{M}}{ⅆ t} , wherein$$

C=a predetermined constant in Ohm.cm, L=the shortest distance between said one or more upper measuring electrodes and said one or more lower measuring electrodes in cm, when the device is being used, M₀=basic myocardial impedance in Ohm, DFT=said duration of said diastolic filling time of said heart in seconds, and

$$Δ \frac{ⅆ {Z}_{M}}{ⅆ t} =$$

is said difference of the value of said first time derivative of said impedance signal between the beginning and the end of said pre-ejection period of said heartbeat of said heart in Ohm/s, wherein the equation M₀=factor·Z₀ holds, wherein
Z₀=is the value of said impedance signal at the end of said pre-ejection period, and factor=a number with a value of 0.54±0.02. Such a device offers very good and accurate results, as experiments have shown. The factor may be considered a geometrical calibration factor, which may depend on electrode configuration and needs to be determined only once for every configuration and patient. The factor will be elucidated lateron.

Preferably, C equals the resistivity ρ of the blood of said mammal at the frequency of the applied alternating current. This has been shown to give highly correlated results when determining LVEDV. The resistivity ρ may be calculated with the formula: C=ρ·53.2·e^{0.022·Hct} in Ohm.cm. Herein, Hct=hematocrit value of a sample of the mammal's blood. Since in hospitals it is almost standard practice to take a blood sample, this a not very bothersome for the patient. It should be noted that the hematocrit value may vary from patient to patient, but also in a patient during the course of time. Preferably, therefore, from time to time a new value for Hct should be determined. Advantageously, this is done every two to three hours, and especially whenever an action is performed which directly influences the Hct-value, such as administering or extracting blood fluid, when diuretics are taken, etc.

Advantageously, the processing unit further can output the ejection fraction EF as

$$EF = \frac{SV}{LVEDV} , wherein$$

SV=the instantaneous stroke volume of said heart, as determined in a manner known per se, and LVEDV=said left ventricular end diastolic volume. Preferably, the SV is determined using formula (1), but such that Z₀ is determined as the actual impedance value at the time of (dZ/dt)max. This has been shown to give more accurate results, that correlate better to clinical standards. This way, the functionality of the device is increased in that it can determine and output an additional important quantity, using techniques known in itself, and to be discussed lateron.

In a preferred embodiment of the device according to the invention, each of the measuring electrodes and/or the supply electrodes have been chosen from the group consisting of strip electrodes and spot electrodes. Strip electrodes give a fairly even current distribution, and clean and clear measuring signals. They may however be somewhat difficult to apply. Spot electrodes are easily applied, but may give rise to more pronounced edge effects and other current inhomogeneities. If desired a combination may be applied, to make use of the advantages of either type of electrodes.

Another aspect of the present invention is a method for determining at least the left ventricular end diastolic volume LVEDV for a heartbeat of an intermittently contracting heart of a mammal, in particular a human being, having a body having a neck and having a thorax having a sternum. The method comprises the steps of applying to the body one or more upper measuring electrodes, one or more lower measuring electrodes, one or more upper supply electrodes and one or more lower supply electrodes, such that the heart is completely between each of the one or more upper measuring electrodes and each of the one or more lower measuring electrodes and such that each of the measuring electrodes is between the one or more upper supply electrodes and the one or more lower supply electrodes, supplying an electrical alternating current with a frequency by means of a current source which is connected to the upper supply electrodes and the lower supply electrodes, receiving an impedance signal which depends on the impedance of a part of the thorax, which part at least comprises the heart, by means of the one or more lower measuring electrodes and the one or more upper measuring electrodes, measuring at least the value of the impedance signal by means of measuring means, and determining the value of the first time derivative of the impedance signal, wherein the method further comprises the step of determining the duration DFT of a diastolic filling time of the heart, and wherein the value of the left ventricular end diastolic volume LVEDV is determined as

$$LVEDV = C \cdot {( \frac{L}{{M}_{0}} )}^{2} \cdot DFT \cdot Δ \frac{ⅆ {Z}_{M}}{ⅆ t}$$

wherein
C=a predetermined constant in Ohm.cm,
L=the shortest distance between the one or more upper measuring electrodes and the one or more lower measuring electrodes in cm,
M₀=basic myocardial impedance in Ohm,
DFT=the duration of the diastolic filling time of the heart in seconds, and

$$Δ \frac{ⅆ {Z}_{M}}{ⅆ t} =$$

is the difference of the value of the first time derivative of the impedance signal between the beginning and the end of a pre-ejection period of said heartbeat of said heart, in Ohm/s, which pre-ejection period is determined in a manner known per se, wherein the equation
M₀=factor·Z₀ holds, wherein
Z₀=is the value of the impedance signal at the end of said pre-ejection period, and
factor=a number with a value equal to 0.54±0.02.

In a preferred embodiment of the method according to the invention, said duration DFT is determined as the time between the moment at which said first time derivative assumes a third local minimum value following a maximum value during a heartbeat, and the moment at which said first time derivative assumes a minimum value immediately before the next maximum value.

In another preferred embodiment of the method according to the invention, said duration DFT is determined by measuring heartsounds of said heart with the help of additional sound recording means, wherein said duration DFT is determined as the time between the beginning of the second component of a heartsound following a maximum value of said first time derivative for said heartbeat, and the beginning of the next heartsound. These are but two methods of measuring the duration DFT. Any other known method to measure this duration DFT would suffice as well. It is not the determination of the duration DFT per se, but the use thereof in determining the left ventricular end diastolic volume, which forms part of the invention.

Preferably, said one or more upper measuring electrodes are applied to the mid neck region and said one or more lower measuring electrodes are applied at the height of the xiphoid junction of the sternum. By thus standardizing the place of application, reliable and accurate results can be obtained. It is important to always measure a standard (large) part of the thorax tissue, always comprising the complete heart.

Advantageously, said constant C is the resistivity of the blood of said mammal at the frequency of the applied alternating current. By thus choosing the value for C, very accurate results have been obtained. Preferably, the resistivity may be determined from the hematocrit value Hct of a sample of the blood of said mammal, using the formula ρ=53.2·e^{0.022·Hct}. Again, advantageously, the hematocrit value Hct is determined every two to three hours, and whenever an action is performed which directly influences the Hct-value, such as administering or extracting blood fluid, when diuretics are taken, etc.

In a preferred embodiment of the method according to the invention, further the ejection fraction EF is determined as

$$EF = \frac{SV}{LVEDV} , wherein$$

SV=the instantaneous stroke volume of said heart, which volume is determined in a manner known per se, and
LVDEV=said left ventricular end diastolic volume. By thus employing known methods to determine SV, the additional important quantity EF may be determined, in order to monitor cardiac function. Preferably, SV is determined using formula (1), however determining Z₀ as the impedance value at the time of (dZ/dt)max. This gives even more accurate results that correlate better to clinical standards.

In the method according to the invention, at least one value chosen from the group consisting of left ventricular end diastolic volume LVEDV and ejection fraction EF, is determined only when the value of said impedance signal Z₀ substantially equals the average value of at least 5, and preferably at least 10 preceding values of said impedance signal Z₀. This way, possible artefacts due to, e.g. respiratory action, are ruled out as much as possible.

### BRIEF DESCRIPTION OF THE DRAWINGS

FIG. 1 shows a diagrammatic view of a device according to the invention, in use on a diagrammatic patient.

FIG. 2 shows diagrammatically an exemplary measured signal, representing AZ, which is the difference in the impedance with respect to the basic value.

FIG. 3 shows the (inverted) first time-derivative of the ΔZ-signal.

FIG. 4 shows a phonocardiagram-signal.

FIG. 5 shows an electrocardiogram-signal.

FIG. 2 through 5 are taken and represented on the same time-axis, i.e. in all four figures the same point in time is represented by the same position on the horizontal axis. This is further indicated by the vertical dashed lines.

### DETAILED DESCRIPTION OF THE PREFERRED EMBODIMENTS

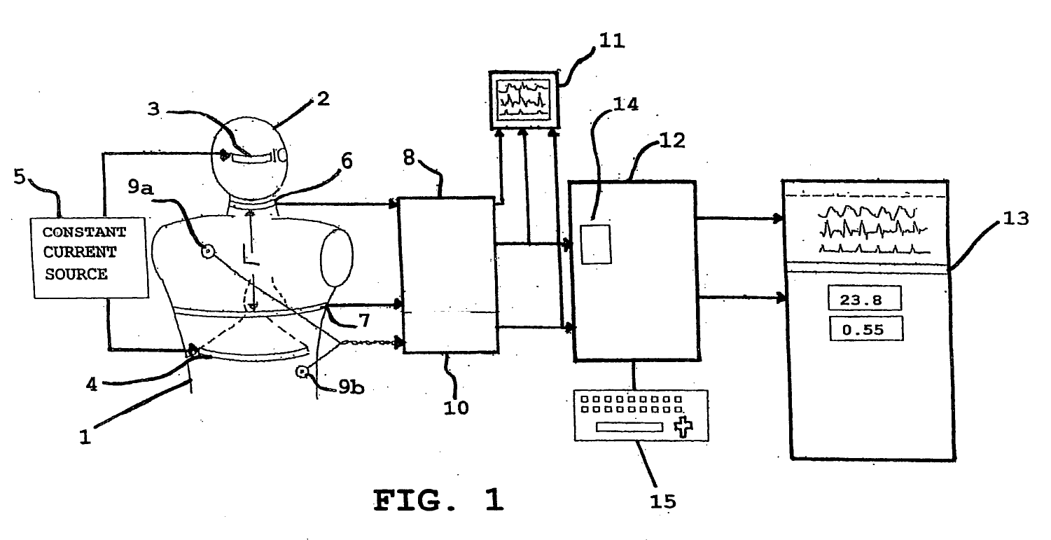

In FIG. 1 there is shown diagrammatically the upper part 1 of the body of a human being, including the head 2.

One or more upper supply electrodes 3 are applied to the forehead, and one or more lower supply electrodes 4 are supplied to the upper part 2 of the body at waist level. A current source 5 is connected to both the upper supply electrodes 3 and the lower supply electrodes 4.

One or more upper measuring electrodes 6 are applied to the body at the midneck region, and one or more lower measuring electrodes 7 are applied at the height of the xiphoid junction of the sternum. The upper 6 and lower measuring electrodes 7 are connected to a measuring device 8.

Furthermore, first 9a and second 9b EKG-electrodes are applied to the body, and are connected to an EKG-measuring means 10.

Both the measuring means 8 and the EKG-measuring means 10 are connected to a display 11 and to a processing unit 12. In its turn, the processing unit 12 is connected to a monitor 13.

The one or more upper supply electrodes 3 and/or the one or more lower supply electrodes 4 are e.g. metallic strip electrodes or spot electrodes. Strip electrodes may be elongate strips passing at least halfway, and preferably all the way around the body of the patient. Spot electrodes may be of a substantially round or square shape, or the like. In the case of strip electrodes, it is preferred to take only one electrode for each of the supply and/or measuring electrodes 3,4,6,7. In the case of spot electrodes, it is possible to use either one, but preferably two or more electrodes, for each of the supply and/or measuring electrodes.

They may be coated with aluminum or some other electrically conducting material. They may be applied to the body with the help of an electrically conducting gel. This not only helps to reduce the transitional impedance between electrodes and skin, but also allows that the patient moves, to some extent, the body part to which the electrodes have been applied, without this having an adverse effect on the measurements.

The supply electrodes 3,4 serve to establish a current field through at least the thorax of the patient. To that end, they should be applied such that at least the thorax, with the heart, is comprised between the upper supply electrodes 3 and the lower supply electrodes 4. This means that the upper supply electrodes should be applied at least as high as the neck level, and the lower supply electrodes at least as low as the level of the xiphoid junction. Preferably however, the upper supply electrodes 3 are supplied at the level of the forehead, because then there will be little or no edge effects and a more homogenous current field. Moreover, there will very likely be no interference with other electrodes or medical apparatus applied to the body of the patient. Preferably, the lower supply electrodes are applied at abdominal level, for equivalent reasons. It should be noted that other heights of application are allowable. However, it may then be necessary to first do a calibration, to correct for possible changes in numerical constants, such as e.g. the factor to determine M₀. Such changes may be due to for example geometrical effects, such as a different current-field through the thorax, caused by a different electrode configuration.

The current source 5 is, although not strictly necessary, preferably a constant current source. This means that the set current remains constant, independent of changes in the impedance through which the current is sent. This greatly improves the accuracy and user-friendliness over non-constant-current sources. Non-constant-current sources may be used if every measurement of the impedance signal Z is corrected for, i.e. divided by, the actual value of the current at the time of the measurement. This is however bothersome, and may decrease accuracy.

The constant current source delivers a current of physiologically safe properties. This means that the used frequency lies in a range within which there is little or no interference with electrical body processes. Advantageously, this frequency range is from about 60 kHz to about 200 kHz, and preferably from about 70 kHz to about 100 kHz. It also means that the current is less than about 5 mA, rms value, and preferably between about 2 and about 4 mA.

The measuring electrodes 6, 7 can be of a type equivalent to the supply electrodes 3, 4, i.e. of the strip type, of the spot type or of mixed type.

The current through the body generates a voltage difference across the body that depends on the magnitude of the current and the impedance of the bodyparts (blood, tissue, etc.) between the electrodes. In fact, Ohm's law is applicable, and the relationship impedance=voltage divided by current may be used to determine the impedance.

By applying the measuring electrodes 6, 7 at the height of the middle neck region and the xiphoid junction of the sternum, respectively, the voltage difference across the thorax can be measured with the help of the measuring means 8. With the help of this voltage signal, the impedance of the thorax, and variations thereof, can be determined. For this reason, and because the voltage signal that is picked up by the measuring electrodes depends on the magnitude of the applied current, the signal is hereinbelow referred to as the impedance signal.

Even though the preferred locations of application are as described above, the measuring electrodes 6,7 may be applied to the body at a different height, but under the following restrictions. Firstly, the thorax with the heart must be comprised between the measuring electrodes 6,7. Secondly, the measuring electrodes 6,7 must be applied between the upper supply electrodes 3 and the lower supply electrodes 4. Thirdly, a calibration may be necessary to account for possible changes in the geometry of the measuring set-up. E.g. if the measuring electrodes are applied to the body somewhat higher or lower, then more or less thorax tissue, blood etc. contributes to the impedance, while the contribution of the heart remains the same. It may then be necessary to redetermine the factor that is used to calculate M₀, by a calibration or correction measurement. This may lead to some changes in the numerical constants that are used in the method according to the invention.

Advantageously, there should be a distance of at least 2 cm between any supply electrode 3,4 and any measuring electrode 6,7 to prevent interference effects and to suppress edge effects. This restriction is more severe in the case of supply electrodes of the spot type than in the case of strip-type supply electrodes. Preferably, this distance is larger, because in persons with short necks, babies, etc. it becomes impossible to place the upper electrodes correctly. Advantageously, the upper supply electrode(s) is/are placed on the forehead. Correspondingly, the lower measuring electrode(s) is/are placed at the abdominal level.

The measuring means 8 can be e.g. a volt-meter or oscilloscope, but any other means to measure a voltage difference would suffice as well. The measuring means determine the value of the voltage between the upper measuring electrodes 6 and the lower measuring electrodes 7. For the purpose of this invention this voltage signal is referred to as the impedance signal, whether or not it has been converted to the underlying impedance value. If not, the voltage signal should be divided by the current in a later calculation.

The measuring means 8 may be connected to an optional display 11. This display 11 can show the measured impedance signal as a function of time. With the help of the display 11 the person operating the device according to the invention can see if the measured impedance signal can be relied upon, i.e. it does not show artefacts. For example, it would be possible for there to be a loose connection or noise. This could result in a measurable but useless signal or a signal with spikes or other physiologically meaningless features. This is not always visible when only a read-out of the value of the impedance signal is considered, but much more easily recognized when looking at the display 11. It should be noted that whenever in this document the term “output”, “outputting” etc. is used, this may mean the displaying on a screen, display, etc., but also the outputting of a signal or value to some further device, e.g. for storage or further processing.

Reference numerals 9a and 9b indicate two optional electrocardiogram (EKG)-electrodes, which are connected to EKG-measuring means 10. In FIG. 1 the EKG-electrodes 9a and 9b are applied to the body in a so-called lead II configuration. They serve to measure the electrical activity of the heart, and can be of any normally used type. The EKG measuring means 10 can be connected to the display 11, in order to be able to visually check the electrical activity of the heart. The EKG-signals as thus determined may be used for several purposes. For example, they can be used for timing purposes, that is, to assign certain signals or points in time to a certain part of the cardiac cycle. Furthermore, it is possible and preferred to establish the heart rate from the EKG-signal. It must be stressed however, that the EKG is not a necessary part of the device according to the invention. The heart rate, e.g., may also be determined from the impedance signal, or by acoustic means.

The measuring means 8, and optionally the EKG-measuring means as well, can be connected to the processing unit 12. Basically, the processing unit 12 is a computer with a computer programme. The processing unit may however come in the form of an electronic circuit or the like, that has been programmed to carry out the method according to the invention. The processing unit 12 may comprise convertor means to sample and digitize the measured impedance signal and turn the analog signal into a digital signal which can be processed by the computer. An analog way of processing the signals is however possible too, though it is very much less flexible, e.g. as to entering of patient related data.

The processing unit 12 further comprises an optional differentiator 14. The differentiator determines the first derivative with respect to time of the measured impedance signal Z, which first time-derivative signal is referred to as dZ/dt. This can either be performed on the analog impedance signal in an analog way by a suitable circuit, or on the digitized impedance signal in a digital way by a suitable programme.

In the preferred embodiment, the device as a whole comprises, as is customary, an invertor which inverts the signal dZ/dt. It is expressly stated here that wherever a maximum or a minimum value of the signal dZ/dt is mentioned, this is meant to be a maximum or minimum value of the inverted signal dZ/dt. In reality this corresponds with a minimum value, a maximum value of the first time-derivative, respectively. If no invertor is used, that is, if the non-inverted signal dZ/dt is used to do the calculations according to the method of the invention, it should be borne in mind that the words “maximum” and “minimum” in the claims should be interchanged. It may be possible to show and/or measure the impedance signal as a difference ΔZ from an average value Z_{avg}. In this case it is possible to invert only ΔZ, after which the signal dZ/dt is already inverted with respect to the real value thereof.

The signal dZ/dt, whether or not inverted, may be displayed on a monitor 13. Not only may the monitor 13 serve as a visual check of the quality of the signals, as stated above in connection with the other signals, but it is also possible to perform the method of the invention by hand on the measured and displayed or output signals. For this purpose it is convenient to connect the display unit with a recording device. This may be e.g. a strip chart recorder, which records one or more of the following signals: impedance signal Z, first time-derivative dZ/dt, phonocardiogram, EKG-signal. However, the full advantage of the method according to the invention may be utilized if it is automated, which is actually done with the help of the device according to the invention. Furthermore, the relevant data can hardly or not at all be determined with the required accuracy, if done by hand.

The processing unit may also comprise data entering means 15. Like in most computers, the data entering means may be a keyboard, a disk drive, a network connection, a modem, or the like. The data entering means may be used to enter data concerning the patient and/or the measurements. As non-limiting examples, the following data may be entered: the shortest distance between the upper and lower measuring electrodes 6,7, the blood resistivity, and patient related data such as name, age, sex, etc.

Furthermore, through the data entering means it is possible to enter computer programmes, such as a programme to determine the stroke volume SV according to any known method, as described earlier. Such programme may also calculate and determine e.g. cardiac output CO(CO=SC×HR), the left ventricular end systolic volume LVESV (LVESV=LVEDV−SV), etc. Also, a modification of the method according to the invention may be entered, e.g. if a calibration is needed if one or more of the electrodes can not be applied to the patient as described in the preferred embodiment.

The monitor 13 may also be used to display the measured values of the left ventricular end diastolic volume LVEDV, and any other quantity measured with the device or method, such as the ejection fraction, the end systolic volume etcetera.

The method and further parts of the device according to the invention will be explained in connection with FIG. 2 through 5.

In FIG. 2 there is displayed an exemplary measured signal indicative of the impedance signal Z. More precisely, FIG. 2 represents the difference ΔZ between the actually measured value of Z and a mean value Z_{avg}, after which the value of ΔZ has been inverted. In practice, the measured signal Z depends for the greater part on the impedance of the respiratory system (mostly the lung tissue), onto which the more rapidly changing impedance of the heart is superposed. Thus, to improve the accuracy of hand performed measurements and to be better able to perform a visual check on the signals, only AZ is shown. But this is not necessary, and it is possible to show the full signal Z.

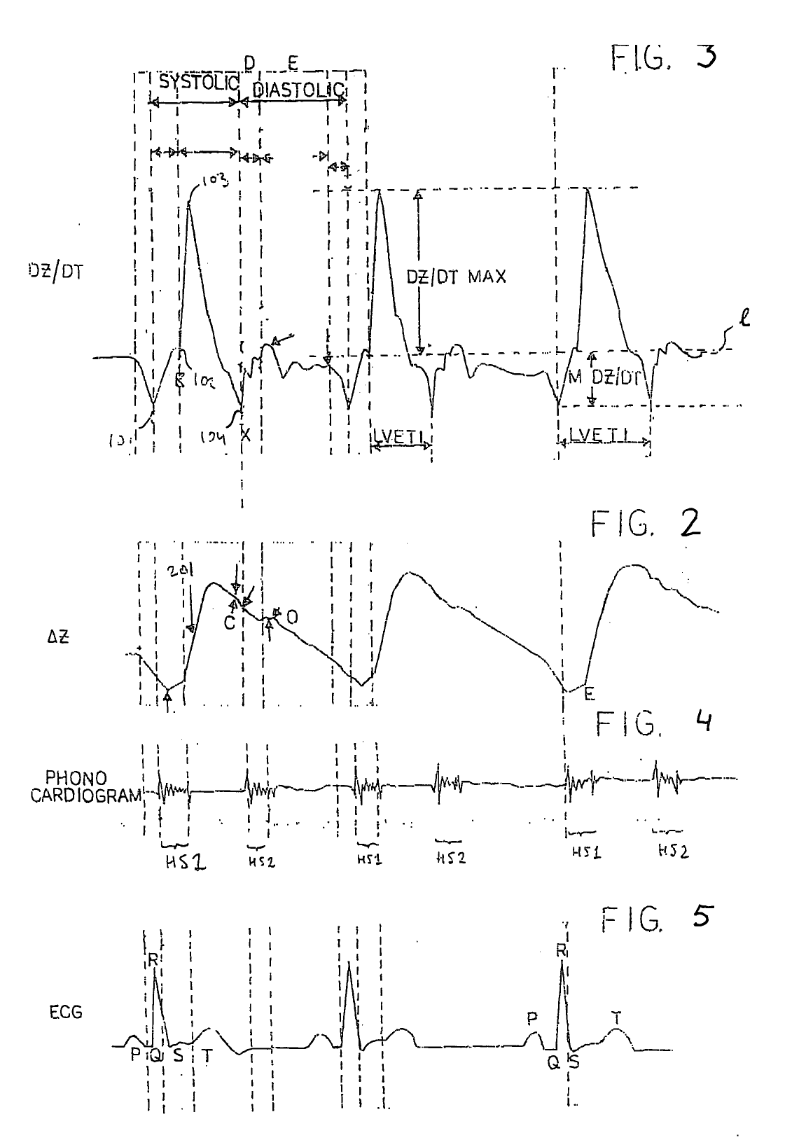

FIG. 3 shows the first time-derivative dZ/dt of the signal AZ. Here, since AZ had already been inverted, dZ/dt as well is inverted with respect to the actual value. This is done out of custom. A reason could be that it looks more natural if a higher peak signal on a screen or display corresponds with a higher LVEDV (or SV) value, even though in reality it is actually a more negative, hence lower value that corresponds with said higher LVEDV value.

FIG. 4 shows a phonocardiogram, taken with the help of a sound recorder, such as a microphone, and an amplifier. Indicated are the two main sounds, viz. the first heartsound HS1 and the second heartsound HS2.

FIG. 5 shows a typical electrocardiogram (EKG), determined with the help of the EKG-electrodes 9a and 9b and the EKG-measuring means 10. Characteristic peaks in the electrical activity of the heart are visible.

With the help of the signals depicted in the FIG. 2-5 various quantities may be determined, as follows from the method of the invention. The most important characteristics will now be elucidated.

Firstly, in the dZ/dt signal many characteristic features may be indicated. These features basically coincide with the corresponding features in the Z-signal diagram, but there they are much less visible.

The period of a heartbeat does not have a definite beginning or end, but for the purpose of this document a heartbeat is said to start at the beginning of the so-called systolic interval, which marks the contracting phase of the heartbeat. This interval starts with the closing of the mitral valve and the tricuspid valve of the heart. The beginning of the closing of these valves is indicated by both the beginning of the first heartsound HS1 in FIG. 4 and the minimum point 101 in FIG. 3. The point 101 is about as much negative as point 104, to be discussed lateron. Point 104 should not be confused with point 102, which is only a (very) local minimum value, not more than a wiggle around the baseline.

The period in the systolic interval that runs from the closure of the mitral and tricuspid valves at point 101 until the opening of the aortic valve is called preejection period, or PEP. Both the point 102 in FIG. 3 and the end of HS1 in FIG. 4 mark the end of the PEP.

During the PEP the heart contracts further and further, and builds up pressure to counteract the closing force across the aortic valve. This closing force is caused by the pressure difference between pressure in the left ventricle and the diastolic pressure in the aorta above the valve. Upon the opening of the aortic valve the heart can eject blood from the left ventricle into the aorta. This forceful ejection of blood causes a steep change in the measured impedance signal Z, which is represented by the slope around point 201 in FIG. 2, and by the peak in the dZ/dt signal around point 103 in FIG. 3. The systolic interval ends with the closure of the aortic valve. This point in time is characterized by the beginning of the second heartsound HS 2 in FIG. 4, as well as with the peak in dZ/dt at point 104.

At the time the systolic interval ends, the diastolic interval begins. At first, the heart muscle relaxes, without any blood leaving or entering the heart. This is called the isovolumetric relaxation period. This period runs from point 104 to point 105. During the isovolumetric relaxation period two peaks 110, 112 are visible. Said isovolumetric relaxation period ends with the beginning of the third peak at point 105. At this time the mitral valve opens, allowing the (left) ventricle to fill with blood. The opening of the mitral valve is marked by the second component HS2-2 of the second heartsound HS2. This filling with blood continues until the closure of the mitral valve at point 101′, with the beginning of the next heartbeat. The closure of the mitral valve is marked by the beginning of the next first heartsound HS1′. The diastolic filling time DFT hence runs from point 105 to point 101′, as well as from the second component of the second heartsound HS2-2 to the beginning of the next first heartsound HS1′. The duration of the diastolic filling time DFT may thus be determined with the help of any means capable of on the one hand measuring time and either determining the point in time at which dZ/dt reaches a certain local minimum value or determining when a heartsound or component thereof begins. Preferably, the means for determining said duration DFT comprise an electronic circuit, which is able to determine said duration DFT as the moment between the moment at which said first time derivative assumes a third local minimum value following a maximum value, and the moment at which said first time derivative assumes a minimum value immediately before the next maximum value. In another preferred embodiment, the means for determining said duration DFT are sound recording means which are able to determine said duration DFT as the time between the beginning of the second component of the heartsound immediately following a maximum value of said first time derivative, and the beginning of the next heartsound. As non-limiting examples, this may be performed by a suitable computer programme for local minimization and/or maximization of the first time derivative, or a triggering device, respectively. Determination of DFT may be performed by hand as well. It should be noted that, in all of this application, the term “maximum” refers to an absolute maximum value during a heartbeat, and “minimum” refers to one of several local minimum values during a heartbeat.

In FIG. 3, line 1 is indicated with the dashed line, and represents the base line, or zero-line. On average, the area subtended by the dZ/dt-signal above the base line 1 will be as large as the area subtended under the base line 1, because the mean impedance as measured over a long period of time will not change. However, mainly because of the influence of the respiratory action, the impedance signal Z, and hence the signal dZ/dt, will undergo a relatively slow, cyclic change around the average, i.e. dZ/dt will change around the base line 1. Keeping this in mind, the following can be said about the determination of the various required quantities.

The quantity M₀ represents the basic myocardial impedance. This quantity may be derived with the aid of equivalent circuit theory in which the basal impedance of the heart M₀ is calculated using a model for the impedance properties of the tissue of the thorax, the heart, the aorta and the two lungs. From this relationship the quantity M₀ has been calculated to be (54±3.2)% of the total basic transthoracic impedance Z. If desired, an adapted model may be adopted to calculate this ratio between myocardial impedance and transthoracic impedance, but the bottom line is that this ratio can be used in the determination of at least the left ventricular end diastolic volume. A possible alternative method to determine M₀ is placing an impedance detection electrode in the right heart, or inserting an impedance electrode down the esophagus and positioning the inner two electrodes above and below the heart. Both these alternative methods suffer however from being invasive.

In fact, in experiments it was shown that when the aorta was blocked, e.g. with the help of an inflatable balloon, the peak 103 completely disappeared, leaving a horizontal line that started at point 102 until it crossed the descending line of the peak 103, where it continued towards point 104. This showed that the aortic part and the myocardial part of the impedance signal could be separated. With this knowledge, their relative magnitudes could be determined using for example the equivalent circuit model.

The quantity Δ(dZ_{M}/dt) represents the change of the dZ/dt-signal that is connected to the myocardium, during the PEP. It is equal to the value of dZ/dt at point 102 minus the value of dZ/dt at point 101. Only in the case that point 102 is on the base line 1, the quantity Δ(dZ_{M}/dt) may be equalled to the absolute value of the value of dZ/dt at point 101. For most healthy patients this will on average be the case, but the respiratory cycle, or physiological anomalies may cause point 102 to lie above or below the base line 1.

However, for the determination of the stroke volume SV, the maximum value of dZ/dt, i.e. the value at point 103, is determined with respect to the base line 1, whether or not point 102 is on the base line 1. It should be noted that a more precise value for the SV may be obtained by taking signals that are not displaced with respect to the base line 1 due to respiratory influences. In most healthy patients, these signals would have the point 102 lying on the base line 1. In patients with physiological anomalies, however, even in that case the point 102 might not lie on the base line 1.

As soon as all relevant quantities have been determined, viz.
the constant C, e.g. from the blood resistivity or hematocrit value,
the shortest distance L between the measuring electrodes 6 and 7,
the basic myocardial impedance M₀,
the total diastolic filling time DFT, and
the change Δ(dZ_{M}/dt) of the first time-derivative of the myocardial impedance, during the preejection period, the device according to the method carries out the required calculation, following the scheme of the method of the invention, and outputs at least the value of the left ventricular end diastolic volume LVEDV.

If desired, other quantities may be output as well, such as the stroke volume SV, ejection fraction EF, which is equal to stroke volume divided by LVEDV, etc. Needless to say, according to the method, the determination of the various quantities may be carried out by hand.

In the method according to the invention, and in using the device according to the invention, the shortest current distance between the measuring electrodes is to be used. Furthermore, the influence of the respiratory movements on the impedance signals is eliminated by preferably only taking signals that have substantially not been displaced with respect to the baseline. In other words, when the instantaneous value of the basic transthoracic impedance Z₀ at the end of the PEP substantially equals the average value of the last 5, advantageously the last 10 measurements of that impedance. This is not to say that the values for LVEDV, EF etcetera cannot be determined when the instantaneous value of Z₀ at the end of the PEP does differ by more than say 5% of the average value, but the results will be less reliable. This may be overcome by averaging the measured values over said 5, 10 or even more heart cycles. This way, the measured values may be better compared with thermodilution values and other values.

As was mentioned in the discussion of the background of the invention, it is repeated here that devices are known which incorporate a constant current source, supply electrodes, measuring electrodes, measuring means and some kind of processing unit, which device is able to determine the stroke volume SV according to the bioimpedance method. The device according to the invention, however, expands the application of the bioimpedance method to the noninvasive, continuous beat-to-beat monitoring of other heart-related quantities as well. Therefore, it could be contemplated that existing devices receive an update in the form of an adapted computer programme, an additional computer programme or an extension unit which is capable of carrying out the method according to the invention. These existing systems should be accurate enough to be able to determine the required data points, e.g. points 101-105, 101′ . . . of FIG. 3.

Various other modifications of the disclosed embodiments of the invention will become apparent to persons skilled in the art upon reference to the description. It is therefore contemplated that the appended claims will cover such modifications or embodiments as fall within the true scope of the invention.

1. Device for determining at least a left ventricular end diastolic volume LVEDV for a heartbeat of a beating heart of a mammal, in particular a human being, having a body having a thorax, the device comprising
a current source for producing an alternating current with a frequency
one or more upper supply electrodes and one or more lower supply electrodes, which can be applied to said body of said mammal and which can be connected to said current source, for supplying said alternating current to said body,
one or more upper measuring electrodes and one or more lower measuring electrodes, which can be applied to said body of said mammal above said heart, below said heart, respectively, for receiving an impedance signal that depends on the impedance of a part of said thorax at least comprising said heart,
measuring means, connected to the measuring electrodes, for measuring said impedance signal which is received by the measuring electrodes,
a processing unit, connected to said measuring means, for processing and outputting at least a value of said impedance signal and a first time derivative of said impedance signal,
wherein the device further comprises means for determining a duration DFT of a diastolic filling time of said heart during said heartbeat and for determining a value of said impedance signal at the end of a pre-ejection period of said heart during said heartbeat, which diastolic filling time and pre-ejection period may be determined in a manner known per se, and wherein said processing unit is designed for determining and outputting said value of said left ventricular end diastolic volume LVEDV in dependence of both said duration DFT and said value of said impedance signal and the difference of said value of said first time derivative of said impedance signal between the beginning and the end of said pre-ejection period.

2. Device according to claim 1, wherein the means for determining said duration DFT comprise an electronic circuit, which is able to determine said duration DFT as the time between the moment at which said first time derivative assumes a third local minimum value following a maximum value during said heartbeat, and the moment at which said first time derivative assumes a local minimum value immediately before the next maximum value.

3. Device according to claim 1, wherein said means for determining said duration DFT are sound recording means which are able to determine said duration DFT as the time between the beginning of the second component of the heartsound following a maximum value of said first time derivative during said heartbeat and the beginning of the next heartsound.

4. Device according to claim 1, in which said processing unit outputs said left ventricular end diastolic volume LVEDV as
$$LVEDV = C \cdot {( \frac{L}{{M}_{0}} )}^{2} \cdot DFT \cdot Δ \frac{ⅆ {Z}_{M}}{ⅆ t} , wherein$$
C=a predetermined constant in Ohm.cm,
L=the shortest distance between said one or more upper measuring electrodes and said one or more lower measuring electrodes in cm, when the device is being used,
M₀=basic myocardial impedance in Ohm,
DFT=said duration of said diastolic filling time of said heartbeat in seconds, and
$$Δ \frac{ⅆ {Z}_{M}}{ⅆ t} =$$
is said difference of said value of said first time derivative of said impedance signal between the beginning and the end of said pre-ejection period of said heartbeat of said heart in Ohm/s, wherein the equation
M₀=factor·Z₀ holds, wherein
Z₀=is the value of said impedance signal at the end of said pre-ejection period, and
factor=a number with a value of 0.54±0.02.

5. Device according to claim 4, wherein C equals the resistivity of the blood of said mammal at the frequency of the applied alternating current.

6. Device according to claim 1, wherein the processing unit further can output the ejection fraction EF as
$$EF = \frac{SV}{LVEDV} , wherein ,$$
SV=the instantaneous stroke volume of said heart, as determined in a manner known per se, and
LVEDV=said left ventricular end diastolic volume.

7. Device according to claim 1, wherein each of the measuring electrodes and/or the supply electrodes have been chosen from the group consisting of strip electrodes and spot electrodes.

8. Method for determining at least a left ventricular end diastolic volume LVEDV for a heartbeat of an intermittently contracting heart of a mammal, in particular a human being, having a body having a neck and having a thorax having a sternum, comprising the following steps
applying to said body one or more upper measuring electrodes, one or more lower measuring electrodes, one or more upper supply electrodes and one or more lower supply electrodes, such that said heart is completely between each of said one or more upper measuring electrodes and each of said lower measuring electrodes and such that each of said measuring electrodes is between said one or more upper supply electrodes and said one or more lower supply electrodes,
supplying an electrical alternating current with a frequency by means of a current source which is connected to said upper supply electrodes and said lower supply electrodes,
receiving an impedance signal which depends on an impedance of a part of said thorax, which part at least comprises said heart, by means of said one or more lower measuring electrodes and said one or more upper measuring electrodes,
measuring at least a value of said impedance signal by means of measuring means, and
determining a value of a first time derivative of said impedance signal,
wherein the method further comprises the step of determining a duration DFT of a diastolic filling time for said heartbeat, and wherein said value of said left ventricular end diastolic volume LVEDV is determined as
$$LVEDV = C \cdot {( \frac{L}{{M}_{0}} )}^{2} \cdot DFT \cdot Δ \frac{ⅆ {Z}_{M}}{ⅆ t}$$
wherein
C=a predetermined constant in Ohm.cm,
L=the shortest distance between said one or more upper measuring electrodes and said one or more lower measuring electrodes in cm,
M₀=basic myocardial impedance in Ohm,
DFT=said duration of said diastolic filling time for said heartbeat in seconds, and
$$Δ \frac{ⅆ {Z}_{M}}{ⅆ t} =$$
is said difference of said value of said first time derivative of said impedance signal between the beginning and the end of a pre-ejection period of said heartbeat of said heart, in Ohm/s, which pre-ejection period is determined in a manner known per se, wherein the equation
M₀=factor·Z₀ holds, wherein
Z₀=is the value of said impedance signal at the end of said pre-ejection period of said heartbeat of said heart, and
factor=a number with a value equal to 0.54±0.02.

9. Method according to claim 8, wherein said duration DFT is determined as the time between the moment at which said first time derivative assumes a third local minimum value following a maximum value during said heartbeat, and the moment at which said first time derivative assumes a minimum value immediately before the maximum value of the next heartbeat.

10. Method according to claim 8, wherein said duration DFT is determined by measuring heartsounds of said heart with the help of additional sound recording means, wherein said duration DFT is determined as the time between the beginning of the second component of said heartsound following a maximum value of said first time derivative for said heartbeat, and the beginning of the next heartsound.

11. Method according to claim 8, wherein said one or more upper measuring electrodes are applied to the mid neck region and said one or more lower measuring electrodes are applied at the height of the xiphoid junction of said sternum.

12. Method according to claim 8, wherein said constant C is the resistivity of the blood of said mammal at the frequency of said applied alternating current.

13. Method according to claim 12, wherein said resistivity may be determined from the hematocrit value Hct of a sample of said blood of said mammal, using the formula ρ=53.2·e^{0.022·Hct}.

14. Method according to claim 8, wherein further an ejection fraction EF is determined as
$$EF = \frac{SV}{LVEDV} , wherein$$
SV=the instantaneous stroke volume for said heartbeat, which volume is determined in a manner known per se, and
LVDEV=said left ventricular end diastolic volume.

15. Method according to claim 8, wherein at least one value chosen from the group consisting of left ventricular end diastolic volume LVEDV and ejection fraction EF, is determined only when the value of said impedance signal Z₀ substantially equals the average value of at least 5, and preferably at least 10 preceding values of said impedance signal Z₀.
